(19) United States
(12) Patent Application Publication
(10) Pub. No.: US 2024/0390195 A1
(43) Pub. Date: Nov. 28, 2024

(54) ELASTIC DIAPER ELEMENT WITH AREAS

(71) Applicant: RKW SE, Mannheim (DE)

(72) Inventors: Christoph Willing, Vreden (DE); Robert KOPLIN, Dittenheim (DE); Reinhard EPPING, Gronau (DE)

(73) Assignee: RKW SE, Mannheim (DE)

(21) Appl. No.: 18/693,715

(22) PCT Filed: Jul. 13, 2022
(86) PCT No.: PCT/EP2022/069587

§ 371 Date: Mar. 20, 2024

(30) Foreign Application Priority Data
Sep. 22, 2021 (DE) 10 2021 124 472.2

(51) Int. Cl. A61F 13/49; A61F 13/15

(52) U.S. Cl. CPC A61F 13/4902; A61F 13/15699; A61F 2013/49022

(57) ABSTRACT

An elastic diaper element with an elastic layer and a layer of nonwoven, wherein the diaper element has connecting regions between the elastic layer and the layer of nonwoven. The diaper element has a first area and at least one further area in which the layer of nonwoven has different proportions of connecting regions in a surface-specific manner in order to ensure area-specific elasticity under load.

### CROSS-REFERENCE TO RELATED APPLICATIONS

This application is a 371 National Phase of PCT/EP2022/069587, filed Jul. 13, 2022, which claims priority from German Patent Application No. 10 2021 124 472.2, filed Sep. 22, 2021, both of which are incorporated herein by reference as if fully set forth.

### TECHNICAL FIELD

The invention relates to an elastic diaper element with an elastic layer and a layer of nonwoven, wherein the diaper element has connecting regions between the elastic layer and the layer of nonwoven.

### BACKGROUND

Elastic, stretchy elements are used in diapers to ensure a good fit and tightness. On the one hand, these elastic elements are used as diaper waistbands, also known as “waistbands”, and as elastic closure elements, so-called “back ears” on baby diapers. On the other hand, the elastic elements are used as belts for diaper underpants.

To increase wearing comfort, these diaper elements contain elastic components that ensure restoring force to the shape of the body thanks to the elasticity of the elastic component. For a pleasant haptic feel, the elastic components, which are usually inserted in the form of films, are provided with textile-like cover materials, for example a nonwoven.

Such nonwovens generally have limited elasticity. In the case of the diaper elements, a nonwoven layer can therefore be partially connected to the elastic layer in a corrugated form. The wave troughs are connected to the elastic layer, while the wave peaks protrude away from the elastic layer and enclose a cavity towards the elastic layer.

DE 602 04 588 T2 describes a manufacturing process for a stretchable elastic composite material. Rollers are used which have a plurality of axially spaced, adjacent, circumferentially extending, identically shaped teeth. The spacings between adjacent teeth form recessed grooves of the same configuration extending in the circumferential direction.

DE 689 23 866 T2 describes a diaper with an upper and a lower layer. An elastic band is attached to the elastic layer in the unstretched state. The elastic band is attached to the elastic layer over its entire surface. After stretching and relaxing the elastic band, gathered areas are formed.

EP 217032 B1 relates to a laminate with an elastic material which is connected at spaced-apart points to at least one web to be laid in folds. The elastic material is a non-corrugated, elastic fiber web.

EP 1 807 035 B1 describes a method for producing a corrugated stretch laminate. In this process, an elastic composition is applied in a molten state to a carrier web to form an elastic element. An expansion composite preform is formed by stretching the carrier web. The preform is then stretched. A substrate is connected to the stretched preform to form a corrugated stretch laminate upon relaxation of the stretched preform.

EP 2 024 178 B1 describes a method for producing an elastically stretchable laminate with three layers. The laminate comprises an elastic film and two layers of non-elastic nonwoven. In one variant, a crepe nonwoven is used. A first elastic laminate is connected to a non-elastic nonwoven layer in a stretched state.

U.S. Pat. No. 6,531,207 B1 describes a stretchable, elastic strip or flap which serves as a fastening strip for the closure of garments with a limited service life and absorbent disposable articles such as diapers and incontinence pads, training underpants, diaper liners, sanitary hygiene articles and similar products. The stretchability is achieved, among other things, via differently stretchable areas of a woven layer.

US 2021/0052438 A1 discloses an elastic laminate comprising an elastic film layer and a nonwoven layer, wherein the nonwoven layer is attached to a first surface of the elastic film layer. The laminate has a first elongation zone and a second elongation zone that differ in elongation properties. However, the laminate is subjected to ring rolling for activation, whereby the nonwoven layer is partially destroyed and thus exhibits a so-called fuzziness.

DE 10 2018 130 054 A1 describes an elastic diaper element with an elastic layer and an outer layer made of nonwoven. The diaper element has connecting regions of the outer layers with the elastic layer. The connecting regions comprise surfaces in which there is a form-fit connection between the nonwoven material of the outer layers and the solidified material of the elastic layer.

DE 10 2019 104 225 A1 discloses a stretchable diaper element with an elastic layer and a nonwoven layer. The elastic element has connecting regions between the elastic layer and the nonwoven layer. The nonwoven layer is corrugated in the unstretched state of the diaper element in order to provide areas as a reserve to enable stretching. The connecting regions comprise surfaces in which there is a form-fit connection between the material of the nonwoven layer and the solidified material of the elastic layer.

WO 2021/115642 A1 discloses a stretchable diaper element with an elastic layer and a nonwoven layer. The elastic element has connecting regions between the elastic layer and the layer of nonwoven. The connecting regions have an extension in a preferred direction and interruptions in their extension.

The diapers and diaper elements described are impressive due to their special construction, which allows them to be elastic and stretchy. However, belt elements and diaper waistbands that are not limited to a uniform elasticity are desirable for high wearing comfort and a simultaneous snug fit to the baby or toddler's body.

### SUMMARY

The object of the invention is to provide a diaper element as a diaper waistband and belt application that adapts specifically to the anatomical needs of the diaper wearer. For this purpose, the diaper element should be anatomically designed to meet the requirements. Furthermore, it should have favorable elastic properties and at the same time ensure high tear resistance. In particular, it should be possible to reposition the diaper waistband. The diaper element should be stretchable when force is applied but should also build up sufficient resistance to give the consumer the feeling of a high-quality product. The diaper element should be harmless to health and ecologically sustainable. Furthermore, the diaper element should have a pleasant feel. The diaper element should also prevent liquid from leaking.

According to the invention, this object is ensured by a diaper element, a method and a use according to the subsidiary main claims. Preferred variants can be found in the description, the embodiment examples, the claims and the drawings.

According to the invention, the diaper element comprises a first area and at least one further area, in which the layer of nonwoven has different proportions of connecting regions in a surface-specific manner to ensure area-specific elasticity under load.

A zone is synonymous with the terms region, area and section. The proportion of connecting regions refers to the area of the nonwoven layer that is defined by connecting regions.

Due to the form-fit connection between the elastic layer and the nonwoven layer, the connecting regions are characterized by high strength. In areas with a high proportion of connecting regions, this results in a lower degree of elasticity, whereas in connecting regions with a low proportion of connecting regions, a higher zonal elasticity is achieved.

The area-specific variation in the proportion of connecting regions creates a diaper element that can be better adapted to the anatomical needs of diaper wearers. In this way, an advantageous wearing comfort can be achieved by creating an area with low elasticity and high snugness at the back and areas with high elasticity in the hip area.

In the areas, the area-specific different proportions of connecting regions of the nonwoven layer can be realized by various design measures. These measures can also be combined with each other.

In one variant, the distances between the connecting regions of the first area differ from the distances between the connecting regions of the other area. In the area with the smaller distances, the position of the nonwoven is determined area-specifically by a larger number of connecting regions.

An alternative is to design the height, in particular the wave height of the areas to form a reserve of the nonwoven layer. Higher corrugations lead to a lower proportion of the connecting regions in relation to the nonwoven surface. This design creates an area with greater elasticity.

Furthermore, in an additional variant, the width of the connecting regions in the first area can be designed differently to the other area. Wide connecting regions achieve a higher proportion of connecting regions regardless of the reference of the layers.

Preferably, the proportion of connecting regions in at least one further area is greater than in the first area by a factor of 1.2, preferably by a factor of 1.4, in particular by a factor of 1.6. As a result, the further area is characterized by a lower degree of elasticity and has a pleasant contact behavior without the application of force. Furthermore, the first area in particular has an advantageous elasticity.

Ideally, the average distance between the connecting regions in the first area is more than 2 mm, preferably more than 3 mm, in particular more than 4 mm and/or less than 6 mm, preferably less than 5 mm, in particular less than 4.5 mm. This contributes to the design of the first area with advantageous elasticity.

Furthermore, the average distance between the connecting regions in the wider area is less than 5 mm, preferably less than 4 mm, in particular less than 3 mm and/or more than 0.5 mm, preferably more than 1 mm, in particular more than 1.5 mm. As a result, the wider area has less elasticity and more support.

In a particularly advantageous variant of the invention, the diaper element has areas arranged mirror-symmetrically to one another. Due to the coordinated arrangement of the areas, the elasticity of the diaper element is ideally adapted to the anatomical requirements.

In a particularly advantageous variant of the invention, the diaper element comprises more than two areas which have surface-specific different proportions of connecting regions to ensure area-specific elasticity under load.

In a further variant of the invention, the areas are not clearly separated from one another and merge smoothly into one another. The further area has a larger proportion of connecting regions, the number of connecting regions of the further area being more than 50%, preferably more than 60%, in particular more than 70% of the total number of connecting regions.

In such a variant, the distances between the connecting regions can also be variable and continuously decrease from the first area to the further area from connecting region to connecting region. Preferably, the distance from the first area to the further area could be smaller as a function of the 2nd or 4th degree. The wider area would then be defined by the larger number of connecting regions and would comprise more than 50% of all connecting regions.

In the mirror-symmetrical design of the areas, the diaper element could also be divided in the middle, resulting in two mirror-symmetrical diaper elements to form a diaper. In addition, the diaper element can be variably divided regardless of the number and design of its areas and connected in various ways to form an advantageous and elastic diaper.

It is particularly advantageous if the connecting regions within an area have an extension in a preferred direction, in particular a straight extension.

In an alternative embodiment, the connecting regions have interruptions in their extension. The interruptions in relation to the total area of the connecting regions can be more than 5 percent by area, preferably more than 20 percent by area, in particular more than 30 percent by area.

To produce the elastic diaper element, a process is preferably used in which the corrugated nonwoven layer is pressed into the molten elastic layer with a roller which has elevations. In this embodiment of the invention, a roller is used whose elevations have interruptions.

In order to meet the aesthetic requirements of a high-quality diaper element, the connecting regions within an area have the same width. This emphasizes the high-quality character of the diaper element. The width of the connecting regions can vary from the first area to the other area or areas.

It is particularly advantageous if the connection regions have a width of more than 0.1 mm, preferably more than 0.3 mm, in particular more than 0.5 mm and/or a width of less than 1.5 mm, preferably less than 1.3 mm, in particular less than 0.9 mm.

It has proven to be advantageous if the connecting regions have an extension perpendicular to the pulling direction of the diaper element. This allows the elasticity and stretchability of the diaper element to be advantageously combined with dimensional stability and snugness.

The high-quality character and the aesthetic appearance of the diaper element is emphasized by the fact that the connecting regions within an area have equal distances to each other. The distances are arranged with a very high degree of accuracy, whereby the relative deviation of the distances is less than 5%, preferably less than 4%, in particular less than 3%.

Preferably, the nonwoven layer is corrugated in the unstretched state of the diaper element. A wave in the sense of the invention is a spatially rectilinear periodic oscillation of the nonwoven layer. It is particularly important here that the nonwoven layer only forms a connecting region with the elastic layer in the wave trough, the lowest point of the periodic oscillation. For each area of elastic layer, an even larger area of non-elastic nonwoven layer is available, which can enable the path of externally applied stretching. This provides zones as a reserve to enable stretching.

In a variant of the invention, the corrugated profile of the nonwoven layer can be formed between two rollers which have elevations and depressions, at least one of the two rollers being designed as a comb roller. The elevations of one roller engage in the depressions of the other roller and vice versa.

In an alternative variant of the invention, the corrugated profile of the nonwoven layer is formed between a roller and a further element which, like the roller, also has elevations and depressions. The elevations of the roller engage in the depressions of the element and vice versa. The element extends in an arc to the extruded elastic layer so that the corrugated profile of the nonwoven layer is maintained up to the connection step.

The term “nonwoven” refers to a fabric that can be made from continuous filaments and/or discontinuous fibers without weaving or knitting by processes such as spunbonding, carding or meltblowing. The nonwoven may comprise one or more layers of nonwoven, each layer containing continuous filaments or discontinuous fibers. Nonwoven may also include bicomponent fibers, which may have fiber structures such as sheath/core, side-by-side.

To ensure area-specific elasticity, the areas of the diaper element can have different wave heights of the nonwoven layer. Within an area, the heights of the nonwoven corrugations are completely uniform, creating the visual impression of a very high-quality product. Higher corrugations of the nonwoven layer allow greater expansion of the diaper area and therefore greater elasticity. Lower waves of the nonwoven layer create areas with less elasticity. In combination with the appropriate design of the connecting regions, the area-specific elasticity of the diaper element is designed to correspond to the anatomical needs of the diaper wearer.

Preferably, each area has a uniform height of the nonwoven corrugations. The height of the reserve zones of the nonwoven layer in the first area can differ from the height of the zones in the further area.

In a particularly favorable variant of the invention, the height of the zones in the first area is more than 0.5 mm, preferably more than 1 mm, in particular more than 1.5 mm, and/or is less than 5 mm, preferably less than 4 mm, in particular less than 3 mm. Furthermore, the height of the zones in the further area is more than 3 mm, preferably more than 4 mm, in particular more than 5 mm and/or less than 9 mm, preferably less than 8 mm, in particular less than 7 mm.

Ideally, the reserve zones of the nonwoven layer within an area have the same heights in relation to each other. The relative deviation of the heights is less than 8%, preferably less than 6%, in particular less than 4%.

In a further variant of the invention, the diaper element has areas with different widths of the corrugation of the nonwoven layer, whereby the area-specific elasticity can be varied.

To create a particularly appealing and valuable diaper element, the zones as a reserve within an area have the same distances from each other. This is achieved by particularly precise spacing, in which the standard deviation of the spacing is less than 5%, preferably less than 4%, in particular less than 3%.

In a particularly advantageous variant of the invention, the diaper element has a further layer of nonwoven, the elastic layer being arranged between the two layers of nonwoven.

The term “elastic” preferably refers to any material which, upon application of a directed force, can stretch to an elongated length of at least about 160% of its relaxed original length without tearing or breaking and which, upon release of the applied force, recovers at least about 55% of its elongation, preferably substantially to its original length, i.e. the recovered length is less than about 120%, preferably less than about 110%, preferably less than about 105% of the relaxed original length.

The invention comprises a method for manufacturing an elastic diaper element in which connecting regions are formed between a layer of nonwoven and an elastic layer. In particular, in a first area, the layer of nonwoven is fixed in a surface-specific manner with a different proportion of connecting regions than in a further area. In the design variant with more than two areas, the area-related proportions of the connecting regions vary from area to area. This makes it possible to create diaper elements with area-specific elasticity in order to better meet the anatomical needs of the diaper wearer.

In order to produce the diaper element, the nonwoven layer can be formed into a three-dimensional, corrugated shape before the connection step by guiding it over a special device. This device can be a roller that has elevations and thus forms the corrugated profile of the nonwoven layer. In addition or alternatively, the nonwoven layer can be guided over an element before the connection step, which extends in an arc to the extruded elastic layer, so that the corrugated profile of the nonwoven layer is maintained until the connection step.

The connection regions can be created in different ways. In one variant, the connecting regions are created by ultrasonic welding. In another variant, parts of the nonwoven layer are pressed into the molten elastic layer. As an alternative to molten extrusion, an already solid elastic layer can be unrolled from a film roll and heated using heating rollers to create a connection with the nonwoven.

In a particularly favorable embodiment of the invention, there is a form-fit connection of solidified elastic material and nonwoven material in the connecting regions. In a variant of the invention, the nonwoven material is not melted, but the fibers are merely pressed into the elastic melt. The material of the elastic layer surrounds the filaments of the nonwoven layer, so that after the elastic layer has solidified, a form-fit connection is created in the connecting regions.

The connecting regions according to the invention are preferably formed as strips in rows arranged next to each other. Within a row, there is preferably a straight course. The orientation of the connecting regions is preferably perpendicular to the pulling direction of the diaper element, so that the individual rows are oriented transversely to the pulling direction of the diaper bundle.

Important for an optimal diaper element is the ratio of the raised to the lowered areas of the devices used to form the shaft profile and in the connection step. This ratio is also referred to as the web-to-groove ratio. Preferably this web-to-groove ratio is less than 1:1, preferably less than 1:2.

There is a form-fit connection between the solidified elastic material and the nonwoven material in the connecting regions. The nonwoven material is preferably not melted, but the fibers are merely pressed into the elastic melt. The material of the elastic layer surrounds the filaments of the nonwoven layer, so that after the elastic layer has solidified, a form-fit connection is created in the connecting regions.

Zones are arranged between the connecting regions in which the corrugated nonwoven layer protrudes away from the elastic layer in an undulating manner and encloses cavities. These zones serve as a reserve for the stretching of the diaper element, whereby there is no connection between the nonwoven layer and the elastic layer in these zones.

In a particularly advantageous variant of the invention, the nonwoven layer consists of an elastic nonwoven. This results in a design of the diaper element which has particular elasticity due to the zones as a reserve for stretching and the elastic layer of nonwoven itself.

In relation to the total area of the flat film, the reserve zones have a significantly larger proportion than the connecting regions. Preferably, the proportion of the reserve zones is more than 60%, in particular more than 70%, preferably more than 80% of the total area. The surface of the solidified, flat, elastic layer is used as a reference for the total area.

Due to the undulating design, the nonwoven layer of one area of the diaper element is significantly longer than the elastic layer. Preferably, the nonwoven layer is longer than the elastic layer by more than a factor of 1.5, in particular by more than a factor of 2.0, preferably by more than a factor of 2.5.

In a preferred variant of the invention, the diaper element is stretched in the transverse direction after the connection process. Preferably, the stretching takes place below the elongation at break of the nonwoven layer.

It is particularly advantageous if the non-connecting zones, i.e. the reserve zones, have a width of more than 1.5 mm, preferably more than 2 mm, in particular more than 2.5 mm and/or a width of less than 6 mm, preferably less than 5 mm, in particular less than 4 mm.

Rollers with a surface structure are preferably used to create the connecting regions, whereby the height of the elevations is more than 100 μm, preferably more than 500 μm, in particular more than 1 mm and/or less than 12 mm, preferably less than 10 mm, in particular less than 8 mm.

Preferably, the nonwoven layer consists of a hydroentangled nonwoven. Fibers in the nonwoven can be reoriented by hydroentanglement so that the original two-dimensional fiber orientation is converted into a three-dimensional fiber orientation. The fibers are more strongly integrated into the nonwoven. This layer of nonwoven preferably has a specific weight of 5 to 80 g/m², preferably 10 to 70 g/m², in particular 15 to 35 g/m².

Preferably, the hydroentangled nonwoven layer is a nonwoven made of continuous filaments. Due to their manufacturing process, these provide a fiber pile that is preferably looped.

Spinnable polymers such as polyester, PLA, polyolefins, in particular polypropylene and polyethylene, can be used as the material for manufacturing the continuous filaments.

The use of hydroentangled nonwoven according to the invention as a corrugated layer of nonwoven, which forms reserve zones for stretching the diaper element, is particularly advantageous. The corrugation deforms the hydroentangled nonwoven in such a way that the fibers in the connecting regions are stretched and thus preferably undergo an orientation. This creates particularly advantageous connecting regions in which the molten material surrounds the stretched and aligned filaments of the hydroentangled nonwoven and a particularly favorable form-fit connection is created after solidification. Surprisingly, it was found that the use of a hydroentangled nonwoven creates a diaper element with particularly favorable properties. The nonwoven layer made from the hydroentangled nonwoven can be formed particularly well.

The elastic layer is preferably a polypropylene and/or a polyethylene block copolymer. The elastic film preferably has a specific weight of 5 to 140 g/m², in particular of 10 to 130 g/m², preferably of 20 to 50 g/m².

Alternatively, the elastic layer can also consist of an SBC (styrene block copolymer) or an elastic polyurethane.

In one variant of the invention, the elastic layer has a multi-layer structure and is preferably designed as a coex film. In an advantageous variant, this comprises a core layer and a significantly thinner skin layer. The skin layer preferably has a specific weight of less than 5 g/m², in particular less than 4 g/m², preferably less than 3 g/m² and/or more than 0.3 g/m², in particular more than 0.6 g/m², preferably more than 0.9 g/m².

In one variant, the core layer is embedded between two outer skin layers.

The core layer preferably consists of an elastic polyolefin, an SBC (styrene block polymer) or a polyurethane.

The skin layer preferably consists of a polyethylene, a polypropylene or an EVA (ethylene-vinyl acetate copolymer).

In a favorable variant of the invention, filled polyolefins are used as skin layers. Mineral materials such as calcium carbonate or talcum are used as fillers, for example. The filler content is preferably more than 60% by weight, in particular more than 70% by weight, preferably more than 80% by weight. The core layer can also be filled.

Blends can also be used to form the skin layer. For example, blends of polyolefins with polystyrene and/or blends of polyolefins with PLA are suitable.

The skin layer is designed in such a way that it ensures unblocking from the sticky core layer. The skin layer is also easily deformable and stretches well.

The elastic layer of the diaper element can also be embedded between two layers of nonwoven. Preferably, at least one layer consists of a hydroentangled nonwoven material. The second nonwoven layer can also consist either of a hydroentangled nonwoven material or a carded nonwoven material or a spunbonded nonwoven material. The second nonwoven layer can either be corrugated or have a flat profile.

Due to the design of the diaper element according to the invention, the diaper fits snugly against the body and ensures an optimal fit. Due to the height of its folds, the diaper element has a voluminous design and fills cavities between the diaper and the body, so that leakage is effectively prevented.

Preferably, rollers with parallel webs and grooves are used to produce the diaper element. Circumferentially identical, wavy grooved rollers with ridges and grooves have proven to be particularly advantageous. These preferably have an amplitude of approx. 1 to 6 mm at a frequency of approx. 30 to 100 mm. The rollers, which have wavy grooves with a depth of approx. 0.5 to 10 mm, interlock and roll in opposite directions with identical circumference and identical speed.

The use of rollers with parallel or corrugated webs, whereby the webs have an intermittent height profile in the direction of rotation, proves to be particularly favorable. The arrangement of the webs on the rollers reflects the zonal structure of the diaper element.

According to the invention, the adhesive-free, areal, elastic nonwoven laminate is used as an elastic diaper element, in particular as a diaper waistband or as a belt element in diaper underpants.

Further advantages and features of the invention are apparent from the description of an embodiment example with reference to drawings and from the drawings themselves. It shows:

### BRIEF DESCRIPTION OF THE DRAWINGS

FIG. 1 a sectional view through a diaper element according to the invention,

FIGS. 2A and 2B schematically show two areas of the diaper element,

FIGS. 3A and 3B schematically show two areas of the diaper element,

FIGS. 4A and 4B schematically show two areas of the diaper element,

FIG. 5 a top view of the zone arrangement,

FIG. 6 a top view of an alternative zone arrangement,

FIG. 7 a detailed view of two connecting regions,

FIG. 8 a detailed view of the reserve zone.

### DETAILED DESCRIPTION

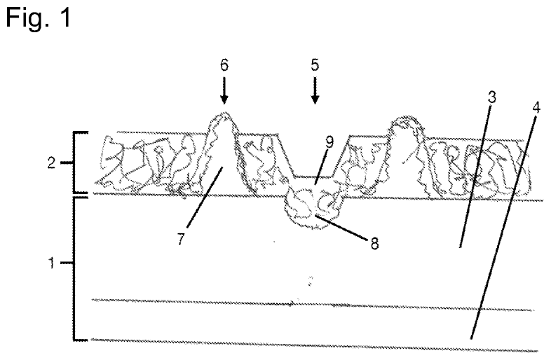

FIG. 1 shows a sectional view through a diaper element according to the invention. The diaper element comprises an elastic layer 1 and a layer 2 of nonwoven made of a corrugated nonwoven. This is a hydroentangled nonwoven, whereby a spunbonded nonwoven made of continuous filaments is used in the embodiment example. In accordance with the invention, the nonwoven is formed into a corrugated shape before being connected to the elastic layer 1. After extruding the elastic layer 1 to form the corrugated nonwoven layer 2, the indentations in the nonwoven are pressed into the molten elastic layer 1, so that connecting regions 5 are formed between the corrugated nonwoven and the elastic layer 1.

In the embodiment example, the elastic layer 1 is shown as a multilayer coex film, with a core layer 3 and a further layer 4, which is designed as a “skin layer”. The ratio of the thickness of the core layer 3 to the further layer 4 is preferably more than 8:1, in particular more than 10:1, in particular more than 12:1: The skin layer 4 preferably has a specific weight of between 1 and 5 g/m².

The core layer 3 is preferably made of thermoplastic polymers. Preferably, polypropylene-polyethylene block copolymers are used, for example of the Exxon Vistamaxx: VM 6102, or VM6202 or VM 7810 and/or the Dow Infuse series: Infuse 9507, Infuse 9107.

The outer layer 4 preferably consists of a polyolefin or an ethylene-vinyl acetate copolymer (EVA). In contrast to the core layer 3, the outer layer 4 is not “sticky” and thus prevents unwanted adhesion.

According to the invention, the laminate comprises connecting regions 5 and reserve zones 6. The reserve zones 6 have no or only a very weak connection with the elastic layer 1 and preferably include cavities 7.

The connecting regions 5 have different zones 8, 9 in the embodiment example.

The outer zone 9 is free of elastic material so that the elastic material does not penetrate the nonwoven layer 2. The nonwoven material in the outer zone 8 is not thermally affected from the outside, so that the filaments of the nonwoven layer 2 are not melted.

In the inner zone 8 of the connecting regions 5, there is a form-fit connection of solidified elastic material and nonwoven material. In the embodiment example, the nonwoven material is also not melted in the inner zone 8. The continuous filaments of the hydroentangled nonwoven are merely pressed into the elastic melt so that a form-fit connection is created after solidification. The continuous filaments of the hydroentangled nonwoven itself remain largely unaffected during the connection process. They are merely surrounded by the molten material of the elastic layer 1.

After the elastic material has solidified, the inner zone 8 has a form-fit connection between the nonwoven material and the solidified material of the elastic layer 1.

The laminate shown in the figure is connected between a pair of rollers, in which a profiled roller with elevations presses the nonwoven layer 2 into the elastic layer 1 from above and a counter roller with a smooth surface is arranged from below. A chill roller is used as a counter roller in the production of the laminate shown in the figure. The chill roller is a steel roller. The roller acting from above is a non-chilled roller.

The rollers used for connecting are moved at a distance, whereby a fixed distance is set.

In the example embodiment, the connecting regions 5 and the reserve zones 6 are designed in the form of strips, with the strips running transverse to the direction of pull of the diaper element.

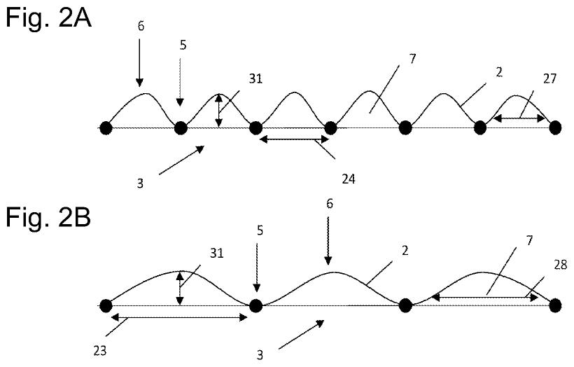

FIGS. 2A and 2B schematically shows two areas of the diaper element according to the invention. FIG. 2A corresponds to the embodiment example in FIG. 1. In this schematic representation, the connecting regions 5 are shown as circular, filled-in dots. The reserve zones 6 are shown as a wave of the layer 2 made of nonwoven. The core layer 3 is arranged underneath. The cavity 7 is enclosed between the corrugation of the reserve zones 6 and the core layer 3 as well as between each two connecting regions 5.

FIG. 2B shows an area of the diaper element with greater elasticity. The distance 23 between two connecting regions 5 is approximately twice as large as the distance 24 in FIG. 2A. In this embodiment example, the distance 23 is 4 mm and the distance 24 is 2 mm. Accordingly, the shaft of the reserve zones 6 is preferably twice as long and the cavity 7 is significantly more voluminous. The shaft height 31 of the reserve zones 6 is identical in both zones shown. The zone of the diaper element in FIG. 2A therefore has a larger area-specific proportion of connecting regions 5. The wave width 28 of the reserve zone 6 is therefore twice as wide as the wave width 27. The area in FIG. 2B is characterized by greater elasticity compared to the area in FIG. 2A, whereby the area in FIG. 2A has greater restoring forces and a greater expansion potential.

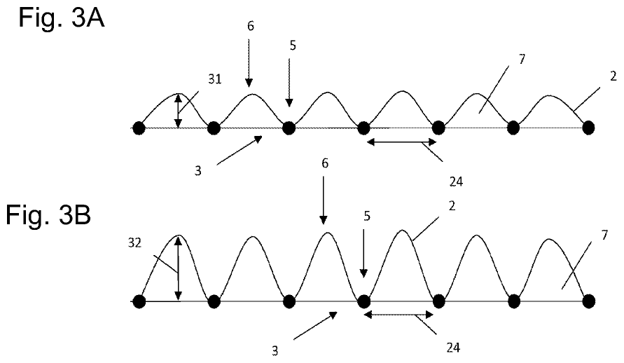

FIGS. 3a and 3B schematically shows two areas of the diaper element according to the invention. FIG. 3A corresponds to the embodiment example in FIG. 1. The distances 24 between the connecting regions 5 are identical. The shaft height 32 in FIG. 3B is greater than the shaft height 31 in FIG. 3A. In this embodiment example, the wave height 32 is 6 mm and the wave height 31 is 2 mm. Due to the arrangement of the connecting regions 5, the area in FIG. 3B has an almost identical elasticity and restoring force as the area in FIG. 3A, but has a much greater expansion potential.

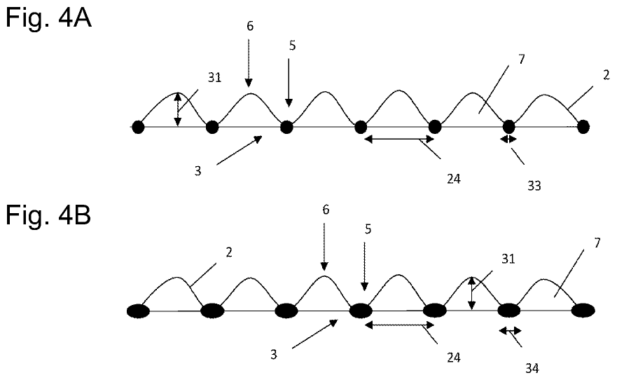

FIGS. 4A and 4B schematically shows two areas of the diaper element according to the invention. FIG. 4A corresponds to the embodiment example in FIG. 1. The shaft height 31 and the distances 24 are identical in FIGS. 4A and 4B. The area in FIG. 4B is characterized by wider connecting regions 5. In this embodiment example, the width 34 of the connecting region 5 in FIG. 4B is twice as wide as the width 33 in FIG. 4A. The area-specific proportion of connecting regions 5 is significantly larger in the embodiment example in FIG. 4B, resulting in a significantly lower elasticity of the area of the diaper element.

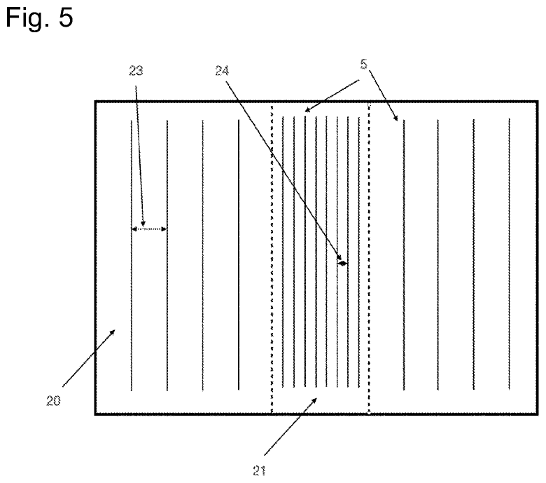

FIG. 5 shows a top view of the area arrangement. Area 20 surrounds area 21 mirror-symmetrically. In area 21, the distances 24 between the connecting regions are shorter than the distances 23 between the connecting regions 5 in area 20. As a result, area 20 has a smaller area-specific proportion of connecting regions 5 and provides greater elasticity than area 21.

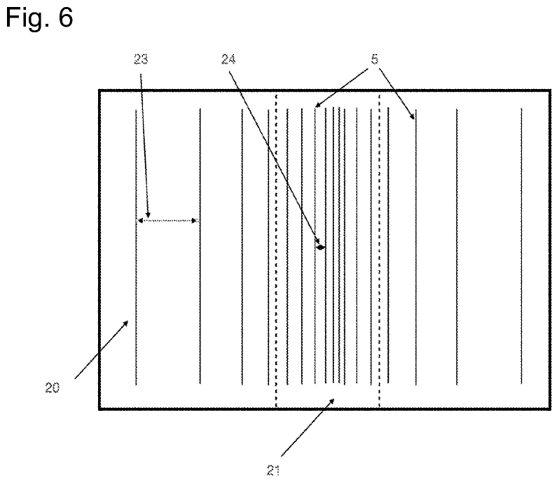

FIG. 6 shows a top view of an area arrangement in which the distances 23, 24 are variable. In this embodiment, the distances 23 from the first area 20 to the distances 24 of the further area 21 become continuously smaller. As a result, the areas 20, 21 are no longer clearly distinguishable, whereby the further area 21 is defined by having more than 50%, preferably more than 60%, in particular more than 70% of the total number of connecting regions 5. In the variant shown in FIG. 6, the further area 21 comprises nine of the seventeen connecting regions 5.

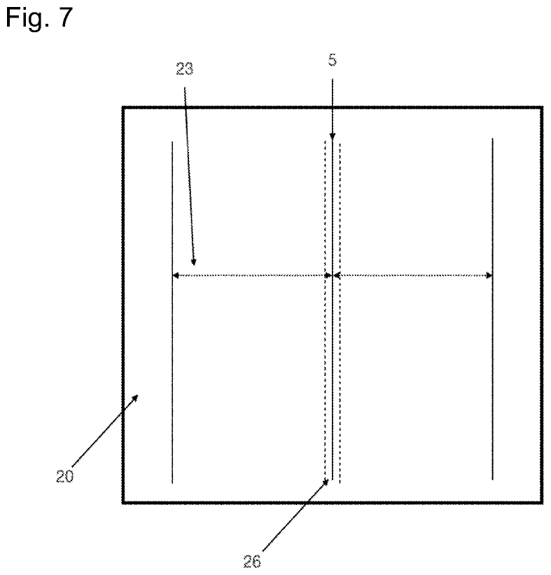

FIG. 7 shows a detailed view of two connecting regions 5. The diaper element is characterized by a high value and an appealing appearance, which is preferably characterized by a particularly precise and detailed design of the connecting regions 5 and their distances 23. The distances 23 are formed with a very high degree of accuracy, whereby the relative deviation 26 of the distances 23 is less than 5%, preferably less than 4%, in particular less than 3%.

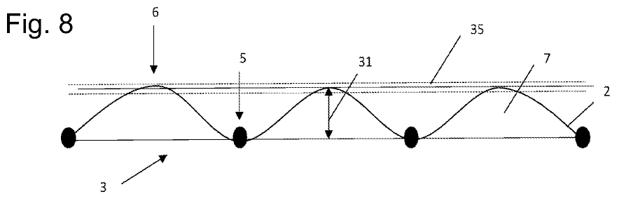

FIG. 8 shows a detailed view of the connecting regions 5 and the reserve zones 6. The diaper element is characterized by a high quality and an appealing appearance, which is preferably characterized by a particularly precise and detailed design of the waves of the layer 2 made of nonwoven and their height 31. The heights 31 are formed with a very high accuracy, whereby the relative deviation 35 of the heights 31 is less than 8%, preferably less than 6%, in particular less than 4%.

1. An elastic diaper element, comprising
an elastic layer (1);
a layer (2) of nonwoven;
connecting regions (5) between the elastic layer (1) and
the layer (2) of nonwoven; and
a first area (20); and
at least one further area (21) in which the layer (2) of nonwoven has different proportions of connecting regions that are area-specific in order to ensure area-specific elasticity under load.

2. The diaper element according to claim 1, wherein a distance (23) of the connecting regions (5) in the first area (20) differs from a distance (24) of the connecting regions (5) of the at least one further area (21).

3. The diaper element according to claim 1, wherein the proportion of the connecting regions (5) in the at least one further area (21) is greater than in the first area (20) by a factor of at least 1.2.

4. The diaper element according to claim 1, wherein a mean distance (23) between the connecting regions (5) in the first area (20) is more than 2 mm and less than 6 mm.

5. The diaper element according to claim 1, wherein a mean distance (24) between the connecting regions (5) in the at least one further area (21) is less than 5 mm and is more than 0.5 mm.

6. The diaper element according to claim 1, wherein the diaper element comprises two of the first areas (20) arranged mirror-symmetrically about the at least one further area (21).

7. The diaper element according to claim 1, wherein the connecting regions (5) have an extension in a predetermined direction.

8. The diaper element according to claim 7, wherein the connecting regions (5) have interruptions in their extension.

9. The diaper element according to claim 1, wherein connecting regions (5) have an extension perpendicular to a pulling direction of the diaper element.

10. The diaper element according to claim 1, wherein the connecting regions (5) within the first area and the at least one further area (20, 21) have a same width (33, 34), the width (33) of the connecting regions (5) of the first area (20) being different from the width (34) of the connecting regions (5) of the at least one further area (21).

11. The diaper element according to claim 1, wherein the connecting regions (5) within at least one of the first area or the at least one further area (20, 21) have equal distances (23, 24) from one another, a relative deviation (26) of the distances (23, 24) being less than 5%.

12. The diaper element according to claim 1, wherein the layer (2) of nonwoven is corrugated in an unstretched state of the diaper element in order to ensure zones (6) as a reserve to enable stretching.

13. The diaper element according to claim 12, wherein a height (31) of the zones (6) of the first area (20) is different from a height (32) of the zones (6) of the at least one further area (21).

14. The diaper element according to claim 13, wherein the height (31) of the zones (6) in the first area (20) is more than 0.5 mm and is less than 5 mm.

15. The diaper element according to claim 13, wherein the height (32) of the zone (6) in the at least one further area (21) is more than 3 mm, preferably more, and is less than 9 mm.

16. The diaper element according to claim 12, wherein the zone (6) as a reserve within at least one of the first area or the at least one further area (20, 21) have same heights (31, 32) relative to one another, with a relative deviation (35) of the heights (31, 32) being less than 8%.

17. The diaper element according to claim 1, further comprising a further layer (2) of nonwoven, the elastic layer (1) being arranged between the layer of nonwoven (2) and the further layer of nonwoven.

18. The diaper element according to claim 1, wherein the at least one further area (21) comprises more than 50%, of a total number of connecting regions (5).

19. A process for producing an elastic laminate, the process comprising:
forming connecting regions (5) between a layer (2) of nonwoven and an elastic layer (1), wherein in a first area (20), the layer (2) of nonwoven is fixed in an area-specific manner with a different proportion of connecting regions (5) than in at least one further area (21).

20. The process of claim 19, further comprising incorporating the elastic element as an elastic diaper element.
